(19) United States
(12) Patent Application Publication
(10) Pub. No.: US 2026/0041626 A1
(43) Pub. Date: Feb. 12, 2026

(54) ENHANCED MOISTURE RETAINING FORMULATION

(71) Applicant: Paige Burdette, Lemont, IL (US)

(72) Inventor: Paige Burdette, Lemont, IL (US)

(73) Assignee: Paige Burdette, Lemont, IL (US)

(21) Appl. No.: 19/293,230

(22) Filed: Aug. 7, 2025

Related U.S. Application Data
(60) Provisional application No. 63/681,577, filed on Aug. 9, 2024.

(51) Int. Cl. A61K 8/9789; A61K 8/23; A61K 8/34; A61K 8/67; A61K 8/73; A61Q 19/00

(52) U.S. Cl. CPC A61K 8/9789; A61K 8/23; A61K 8/345; A61K 8/676; A61K 8/73; A61K 8/735; A61Q 19/007

(57) ABSTRACT

The present invention relates to a novel formulation of a moisturizing formulation and a method for its production. The formulation is designed to provide enhanced hydration, improved skin elasticity, and long-term stability, making it suitable for all skin types, including sensitive skin. The formulation includes a unique combination of ingredients that synergistically contribute to the formulation's effectiveness. Key components include DI Water, Aloe Barbadensis Leaf, Glycerin, Hyaluronic Acid in both high and low molecular weights, Vitamin C, and a naturally derived preservative, Nipaguard SCE. Additionally, the formulation incorporates Astragalus Membranaceus Root Extract, known for its anti-aging and immune-boosting properties, which enhances the skin's resilience against environmental stressors. The method of production involves a process of mixing ingredients in a specific sequence to ensure stability and prevent premature reactions, with particular attention to the homogeneous distribution of components like Xanthan Gum and the proper dispersion of Hyaluronic Acid. This careful preparation method ensures that the final product not only meets the hydration needs of the skin but also remains effective and stable over time. The invention offers a significant advancement in skincare technology, providing a reliable and safe product that delivers both immediate relief and long-term benefits to skin hydration and health.

### CROSS REFERENCE TO RELATED APPLICATIONS

This application claims the benefit of U.S. Provisional Ser. No. 63/681,577, filed Aug. 9, 2024, entitled “Enhanced Moisture Retaining Formulation,” which is hereby incorporated by reference in its entirety.

### BACKGROUND OF THE INVENTION

The skincare industry has long been challenged by moisturizing formulations that not only provide effective hydration but also maintain stability, safety, and long-term benefits without causing irritation or adverse reactions. Traditional formulations often fall short in one or more of these areas due to various limitations.

One common issue with many moisturizing formulations is the inadequate provision of sustained hydration. While these formulations may initially hydrate the skin, they often fail to lock in moisture over extended periods, leading to quick reversion to dryness. This is particularly problematic in environments with low humidity or for individuals with naturally dry skin. Additionally, many formulations do not penetrate deeply enough, providing only superficial hydration that does not improve skin elasticity or overall health.

Another significant challenge is the stability of the formulation. Many formulations are susceptible to separation, degradation of active ingredients (such as vitamins and antioxidants), or microbial contamination. This instability can reduce the efficacy of the formulation over time and may lead to the development of harmful byproducts. Furthermore, the use of inadequate or harsh preservatives can lead to skin irritation or allergic reactions, compromising the safety and comfort of the user.

Moreover, many formulations contain ingredients that, while effective in treating certain skin conditions, may not be suitable for all skin types, particularly sensitive skin. The use of synthetic ingredients and allergens can trigger adverse reactions, limiting the suitability of these products for a broader audience. It remains desirable therefore to have an improved formulation that addresses the above challenges encountered with other existing attempts.

### SUMMARY OF THE INVENTION

The preferred embodiment of the invention comprises a novel moisturizing formulation designed to provide enhanced hydration, improved skin elasticity, and long-term stability. Embodiments further comprise an associated method of production of such moisturizing formulation. The moisturizing formulation described herein specifically addresses the common shortcomings found in existing attempts, such as transient hydration, instability, and potential irritation, by utilizing a unique combination of ingredients and methodical preparation steps.

The core of the preferred embodiment of invention lies in its novel combination of ingredients, which includes DI Water as the solvent, providing a pure and stable base that enhances the effectiveness of other ingredients without causing irritation. Aloe Barbadensis Leaf and Vitamin C are incorporated for their soothing and antioxidant properties, respectively, contributing to the formulation's ability to improve skin health and appearance. Glycerin and Hyaluronic Acid in both high and low molecular weights ensure deep skin penetration and sustained moisture retention. This dual-weight approach allows for immediate and lasting hydration effects, benefiting the skin's surface and deeper layers. Nipaguard SCE, a naturally derived preservative, ensures the product's longevity and safety by effectively preventing microbial growth without adverse effects. In accordance with the preferred embodiment, the method of mixing the ingredients allows for the benefits provided in association with the novel formulation.

The method of preparing the formulation involves specified steps to integrate these ingredients effectively. These steps include the pre-mixing of certain components to ensure uniform distribution and the sequential addition of phases to maintain stability and prevent premature reactions. The final product is a moisturizing formulation that not only meets the hydration needs of the skin but also remains stable and effective over time.

This embodiment of the invention is particularly beneficial for individuals seeking a reliable and safe skincare product that provides both immediate relief and long-term benefits to skin hydration and health. The formulation is suitable for all skin types, including sensitive skin, making it a versatile addition to daily skincare routines.

### BRIEF DESCRIPTION OF THE FIGURES

FIG. 1 depicts an exemplary application of the moisturizing formulation, showing a moisturizing serum contained within a container and dispensed through a pipette dropper in accordance with an embodiment of the invention.

FIG. 2 depicts a flowchart illustrating the formulation process for preparing the moisturizing formulation, including the sequential steps of combining ingredients in two separate containers, mixing procedures, and the final combination to create the finished formulation in accordance with an embodiment of the invention.

### DETAILED DESCRIPTION OF THE INVENTION

The following detailed description of the invention refers to the accompanying figures that illustrate specific embodiments and applications of the moisturizing formulation and associated methods described herein. The figures are provided to enable those skilled in the art to make and use the invention and should be understood as depicting representative embodiments rather than limiting the scope of the invention. The subsequent paragraphs describe the invention as presented in the figures in accordance with various embodiments, providing enabling disclosure of both the product configuration and manufacturing process that collectively demonstrate the practical implementation and commercial viability of the enhanced moisture retaining formulation. These descriptions are intended to complement and further illustrate the detailed compositional and methodological aspects of the invention previously described, showing how the formulation may be packaged, delivered, and produced according to the disclosed embodiments.

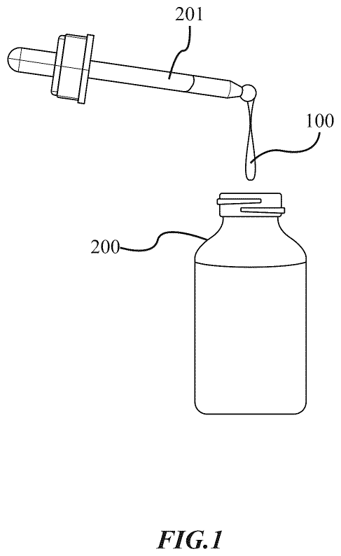

FIG. 1 illustrates the practical implementation and user application of the moisturizing formulation in a commercial packaging configuration. The figure depicts the moisturizing serum (element 100), which represents the finished formulation produced according to the method described herein, contained within a storage container (element 200). The container (200) serves as the primary packaging vessel that maintains the stability and integrity of the formulation throughout its shelf life. The delivery mechanism is shown through a pipette dropper (element 201), which provides precise dispensing control allowing users to measure and apply specific quantities of the formulation. This configuration enables accurate dosing while preventing contamination of the remaining product, as the pipette dropper (201) can be used to extract the desired amount without direct contact between the user's skin and the bulk formulation stored in container (200). The packaging system shown ensures that the formulation's stability is maintained while providing convenient access for topical application to achieve the intended moisturizing and skin health benefits described throughout the specification.

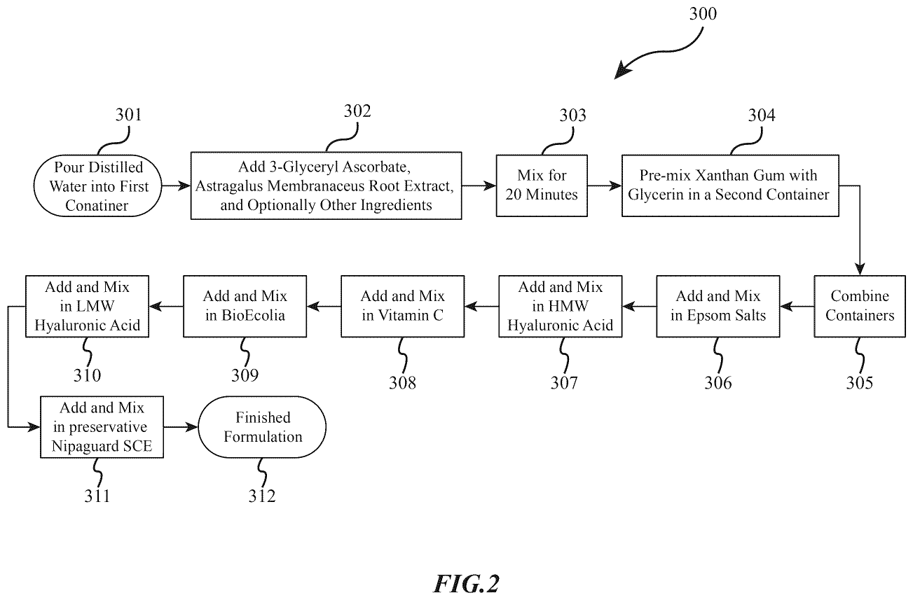

FIG. 2 presents a flowchart illustrating the formulation process (element 300) in accordance with an embodiment of the invention that enables the production of the moisturizing formulation with the precise sequence and timing necessary to achieve optimal stability and efficacy. The process begins with step 301, which involves pouring distilled water into the first container, establishing the aqueous base for the formulation. Step 302 follows with the addition of 3-Glyceryl Ascorbate and Astragalus Membranaceus Root Extract, along with other optional ingredients, to initiate the formation of the active ingredient matrix. A critical mixing period of 20 minutes is implemented in step 303 to allow proper integration and stabilization of these initial components.

The process in accordance with an embodiment continues with step 304, which involves the preparation of a second container where Xanthan Gum is pre-mixed with Glycerin. This pre-mixing step is essential to prevent clumping and ensure homogeneous distribution when later combined with the aqueous phase. The flowchart then details the sequential addition of active ingredients, each followed by a 5-minute mixing period to ensure complete integration: step 305 adds LMW Hyaluronic Acid, step 306 incorporates BioEcolia, step 307 introduces Vitamin C, step 308 adds HMW Hyaluronic Acid, and step 309 includes Epsom Salts. Each 5-minute mixing interval shown in the flowchart is critical in an embodiment for preventing premature reactions and ensuring uniform distribution of each component throughout the formulation.

Step 310 represents the combination of the two containers in accordance with an embodiment, merging the pre-mixed Xanthan Gum/Glycerin mixture with the main formulation. Step 311 involves the addition of the preservative Nipaguard SCE with subsequent mixing to ensure antimicrobial protection throughout the product. The process concludes with step 312, representing the finished formulation ready for packaging. This detailed process flowchart provides the enabling disclosure necessary for skilled practitioners to reproduce the formulation with consistent quality and stability, ensuring that the sequential timing and mixing protocols are followed to achieve the intended therapeutic and cosmetic benefits of the final moisturizing product.

An aspect of the invention comprises a method pertaining to the preparation of a moisturizing formulation. In the preferred embodiment the moisturizing formulation comprises a serum. In alternative embodiments, the moisturizing formulation comprises a lotion or other similar medium. The method is directed to production of a moisturizing formulation formulated to enhance skin hydration and elasticity. This process involves a series of steps designed to integrate various active ingredients effectively, ensuring optimal stability and performance of the final product. The intended output of this method is a moisturizing formulation that provides prolonged hydration, utilizing a blend of natural extracts and scientifically proven moisturizers. The formulation aims to provide added benefits derived from ingredients like Hyaluronic Acid and Vitamin C. Through this method, the formulation not only aims to improve skin moisture retention but also to enhance the overall health and appearance of the skin.

In accordance with the preferred embodiment, the order of combining the ingredients optimizes the resulting mixture in accordance with the method of preparation. The process begins with the addition of DI Water, followed by 3-Glyceryl Ascorbate, and then Astragalus Membranaceus Root Extract. Subsequently, Glycerin, Propanediol, Niacinamide, and Saccharide Isomerate are added. The preservative Nipaguard SCE, which consists of Sorbitan Caprylate, Propanediol, and Benzoic Acid, is then incorporated. Following these, Aloe Barbadensis Leaf, Magnesium Sulfate, Rice Bran Protein, Alpha-Glucan Oligosaccharide, and Sodium Hyaluronate are added to the mixture. Lastly, Xanthan Gum is pre-mixed with Glycerin before being added to ensure a homogeneous distribution. This specific sequence in accordance with the preferred embodiment is critical as it ensures the stability and efficacy of the moisturizing formulation by preventing premature reactions and promoting optimal integration of the ingredients.

A step associated with the method involves adding phase A to the main container, pre-mixing Xanthan Gum with Glycerin before adding to the main container. The initial step involves the preparation of Phase A, which includes combining various ingredients in a specified order. The primary components include DI Water, Aloe Barbadensis Leaf, Glycerin, Xanthan Gum, Magnesium Sulfate, Niacinamide, Propanediol, Hydrolyzed Rice Protein, 3-Glyceryl Ascorbate, Alpha-Glucan Oligosaccharide, and Saccharide Isomerate. A critical aspect of this step is the pre-mixing of Xanthan Gum with Glycerin to ensure a homogeneous distribution before adding to the main container. This pre-mixing is essential to avoid clumping and ensure smooth integration into the aqueous base.

The method also comprises the step of mixing Phase A for at least 20 minutes. During such step, once all ingredients of Phase A are added to the main container, the mixture is stirred continuously for at least 20 minutes. This duration is crucial to allow the ingredients to interact and stabilize, forming a cohesive base for the formulation. The mixing can be performed using mechanical stirrers at a controlled speed to maintain the integrity of sensitive components.

The method further comprises the step of adding phase B in sequence after the preparation of Phase A. During such step, which occurs following the preparation of Phase A, Phase B ingredients are sequentially added to the mixture. These Phase B ingredients comprise Epsom Salts, HMW Hyaluronic Acid, Vitamin C, BioEcolia, LMW Hyaluronic Acid, and a preservative (Nipaguard SCE). In association with such step, each component must be added individually with thorough mixing to ensure complete integration. The order of addition and thorough mixing are critical to prevent premature reactions and to ensure the stability of the final product.

After the introduction of each ingredient, the mixture is subjected to thorough mixing. This is crucial to prevent any premature chemical reactions that could potentially destabilize the mixture. For instance, the careful sequencing and mixing prevent the Vitamin C from degrading due to improper pH levels or interactions with other components.

Moreover, the thorough mixing ensures that the Hyaluronic Acid, both HMW and LMW, is evenly dispersed throughout the formulation. This is essential as Hyaluronic Acid serves as a major hydrating agent, offering sustained moisture retention and enhancing the skin's texture and appearance. The preservative, Nipaguard SCE, is added to maintain the product's integrity and longevity by preventing microbial growth.

The order of addition and the thoroughness of the mixing process are meticulously designed to maintain the stability and efficacy of the final product in accordance with the preferred embodiment. This step not only ensures the chemical stability of the formulation but also enhances its physical properties, such as viscosity and spreadability, making it suitable for consumer use. The detailed attention to the sequence and integration of Phase B ingredients ensures that the final moisturizing formulation meets the desired quality standards and achieves the intended skin health benefits.

The final step in the method comprises mixing the final combination for at least 30 minutes to ensure that the Hyaluronic Acid is properly dispersed. After all components have been added, the final mixture is stirred for an additional 30 minutes. This extended mixing time is vital to ensure that the Hyaluronic Acid, both high and low molecular weight, is fully dispersed throughout the formulation. This step is crucial for achieving the desired texture and ensuring the effectiveness of the moisturizing properties of the final product.

The described process not only ensures the production of a high-quality and differentiated moisturizing formulation but also addresses the stability and efficacy of the product through meticulous ingredient integration and mixing protocols.

An aspect of the invention comprises a moisturizing formulation, in its preferred embodiment produced via the described steps of the aforementioned method. The formulation described provides superior and long-lasting hydration attributes. The moisturizing formulation comprises a carefully selected blend of ingredients, each chosen for their specific properties that contribute to the overall effectiveness of the product. The base of the formulation includes DI Water and Aloe Barbadensis Leaf, each known for their hydrating and soothing properties. The inclusion of Glycerin and Hyaluronic Acid in both high and low molecular weights ensures deep penetration and hydration of the skin, offering both immediate and long-lasting moisture retention. Vitamin C, a potent antioxidant, is incorporated to aid in skin repair and rejuvenation, helping to brighten the skin and reduce the appearance of aging.

DI Water, or deionized water, is water that has had its mineral ions removed, including sodium, calcium, iron, and copper, among others. These ions are removed through a process that typically involves electrically charged resins that attract and bind to the minerals, effectively removing them from the water. In the context of the formulation described herein, DI Water serves as an ideal base or solvent due to its high purity and neutrality. It does not contain any minerals that might interfere with the stability or efficacy of other active ingredients in the formulation. Additionally, because DI Water is free from contaminants and impurities that might be present in regular tap water, it helps in ensuring that the formulation remains gentle and non-irritating to the skin. This makes it particularly beneficial in the context of the formulation described herein, as it supports the maintenance of the product's integrity and safety, while also aiding in the effective delivery of hydrating and nourishing compounds to the skin.

The preferred embodiment of the formulation further comprises Astragalus Membranaceus Root Extract, which is a potent botanical ingredient derived from a traditional herb known for its extensive use in herbal medicine. Chemically, this extract contains various saponins, flavonoids, and polysaccharides, which are compounds known for their anti-inflammatory, antioxidant, and immune-boosting properties. In the context of the formulation, Astragalus Extract contributes uniquely by enhancing the skin's resilience against environmental stressors and aging. The antioxidant properties help in neutralizing free radicals, which are unstable molecules that can damage cells and contribute to premature aging. Additionally, its anti-inflammatory effects can soothe the skin, reducing redness and irritation. By bolstering the skin's defensive barrier, Astragalus Extract not only aids in maintaining moisture and elasticity but also supports overall skin health and vitality, making it a valuable component in skincare formulations aimed at nourishing and revitalizing the skin.

The formulation in its preferred embodiment further comprises Aloe Barbadensis Leaf, commonly known as Aloe Vera, is a naturally derived ingredient renowned for its skin-soothing and healing properties. In the context of a formulation, Aloe Vera provides multiple benefits that enhance the product's effectiveness and appeal. It is rich in vitamins, minerals, antioxidants, and amino acids, which collectively help to moisturize, soothe, and protect the skin. Aloe Vera's hydrating properties are particularly beneficial in a formulation, as it helps to increase the water content in the skin, making it more supple and less prone to dryness and irritation.

Moreover, Aloe Vera has anti-inflammatory properties that can help to reduce skin inflammation, making it ideal for soothing conditions like sunburns or general skin sensitivity. Its gentle nature makes it suitable for all skin types, including sensitive skin. The inclusion of Aloe Barbadensis Leaf in a formulation not only aids in immediate skin relief and hydration but also contributes to long-term skin health by promoting better moisture retention and providing a protective barrier against environmental stressors. This makes it a valuable component in the described formulation aimed at nourishing and revitalizing the skin.

Glycerin and Hyaluronic Acid, both in high and low molecular weights, are key hydrating agents incorporated into the formulation to enhance skin moisture and overall health. Glycerin, a humectant, attracts water from the air and the deeper layers of the skin into the outer layer, providing hydration and a protective barrier that helps prevent moisture loss. This makes the skin appear more supple and smooth. Hyaluronic Acid (HA), available in both high molecular weight (HMW) and low molecular weight (LMW) forms, complements the action of Glycerin by working at different levels of the skin. HMW HA forms a film on the surface of the skin, locking in moisture to provide hydration and reduce the evaporation of water, which is particularly beneficial in dry environments. LMW HA, on the other hand, penetrates deeper into the skin's layers, promoting elasticity and reducing the appearance of fine lines and wrinkles by replenishing moisture from within.

Together, Glycerin and Hyaluronic Acid create a multi-layered hydration effect in the formulation described herein, making them highly effective in both immediate and long-term skin hydration. This combination not only improves the skin's texture and appearance but also enhances the formulation's ability to maintain skin health in various environmental conditions, making it a versatile and essential component in the formulation described herein aimed at delivering deep and lasting hydration.

The use of Xanthan Gum as a thickening agent ensures that the formulation in its preferred embodiment maintains a consistent viscosity that facilitates easy spreading and absorption. This texture makes the formulation suitable for use in various climates and skin types, providing a versatile skincare solution.

Stability is a key aspect of the formulation in the preferred embodiment. The inclusion of a preservative system featuring Nipaguard SCE ensures that the product remains free from microbial growth, thereby extending its shelf life and maintaining its efficacy throughout its usage period. The formulation's pH is carefully adjusted to match that of the skin, ensuring that it is gentle and non-irritating even with regular use. Nipaguard SCE is a preservative used in cosmetic formulations, including formulations, to ensure product safety and longevity. It is a blend of naturally derived ingredients, specifically designed to provide broad-spectrum antimicrobial protection against bacteria, yeast, and molds. In the context of the moisturizing formulation, Nipaguard SCE plays a crucial role in maintaining the integrity and stability of the product throughout its shelf life. The inclusion of Nipaguard SCE in the formulation helps to prevent microbial contamination that can occur during manufacturing, packaging, and consumer use. This is particularly important for products that contain water as they are susceptible to microbial growth. By effectively inhibiting the growth of potentially harmful microorganisms, Nipaguard SCE ensures that the formulation remains safe and effective for use over an extended period. Moreover, Nipaguard SCE is chosen for its compatibility with a wide range of cosmetic ingredients and its efficacy in a variety of pH environments, which is essential for maintaining the effectiveness of other active ingredients in the formulation. Its use in the formulation not only supports the product's safety but also enhances consumer trust and satisfaction by ensuring that the formulation retains its quality and efficacy from the first use to the last.

In an exemplary embodiment of the formulation, the mixture ratios of the ingredients maximize the benefits of hydration, skin health, and stability. The formulation begins with DI Water, which forms the majority of the formulation's base, ensuring adequate solvency and hydration. In the context of the formulation, as the base, the DI Water comprises a substantial part of the total composition, in an embodiment ranging from 70% to 90% of the total formula, depending on the desired consistency and the concentration of other active ingredients. Aloe Barbadensis Leaf is included at 0.10%, providing soothing and healing properties that enhance skin comfort and hydration. Glycerin, at 0.90%, acts as a humectant, drawing moisture into the skin to support hydration and barrier function. Xanthan Gum, used at 0.30%, serves as a thickening agent, helping to stabilize the formulation and improve its texture for easy application.

Magnesium Sulfate is added to an embodiment of the formulation at 0.50% for its skin conditioning properties and to help stabilize the emulsion. Niacinamide, at 2.00%, included in an embodiment of the formulation, enhances the skin's elasticity, improves barrier function, and reduces skin discoloration. Propanediol, included in an embodiment of the formulation at 4.00%, enhances the absorption of ingredients into the skin and offers additional hydration and smoothness. Hydrolyzed Rice Protein, included in an embodiment of the formulation at 0.30%, provides skin conditioning and soothing effects.

A significant component in accordance with a preferred embodiment, 3-Glyceryl Ascorbate or Vitamin C, is incorporated in an embodiment of the formulation at 10.00%, acting as a potent antioxidant that helps to brighten the skin and promote collagen synthesis. In accordance with various embodiments, the inclusion of 3-Glyceryl Ascorbate, which is a highly potent form of Vitamin C, amplifies the efficacy of the Astragalus Root Extract by providing enhanced skin-brightening and collagen-boosting effects. Thus it is a teaching of embodiments of the invention to combine Astragalus Root Extract with 3-Glyceryl Ascorbate within the same composition. The combination of Astragalus Root Extract and 3-Glyceryl Ascorbate offers a powerful synergy that targets multiple signs of aging, improves skin hydration and elasticity, and provides a protective barrier against daily environmental damage. Moreover, 3-Glyceryl Ascorbate serves a dual function by also acting as a natural preservative for the serum. This not only extends the shelf life of the resulting article but does so without the need for synthetic preservatives, which aligns with the growing consumer preference for natural and safe skincare solutions.

Alpha-Glucan Oligosaccharide, included in an embodiment of the formulation at 0.10%, helps to protect and strengthen the skin's natural barrier, while Saccharide Isomerate, included in an embodiment of the formulation at 2.00%, provides deep hydration and lasting moisture retention. The preservative, Nipaguard SCE, is crucial for maintaining the product's integrity and is included in an embodiment of the formulation at 1.00%.

The formulation in the preferred embodiment includes Astragalus Membranaceus Root Extract. In various embodiments, the Astragalus Membranaceus Root Extract comprises 5-15% of the moisturizing formulation. In the preferred embodiment, the Astragalus Membranaceus Root Extract is present in the moisturizing formulation at a concentration of approximately 10.00%. Astragalus Membranaceus Root Extract enhances the skin's ability to defend against environmental stressors, contributing significantly to the formulation's effectiveness. In association with various embodiments, the inclusion of Astragalus Root Extract provides anti-aging and immune-boosting properties, derived from its rich composition of saponins, flavonoids, and polysaccharides. These compounds are known for their potent antioxidant and anti-inflammatory effects, which contribute to the serum's ability to enhance skin resilience against environmental stressors and aging.

Additionally, the formulation in an embodiment contains Sodium Hyaluronate, a sodium salt form of Hyaluronic Acid, which is highly effective at retaining moisture within the skin. Listed at a concentration of 0.30% for LMW Hyaluronic Acid, it aids in deeper penetration into the skin layers. This component is crucial for promoting skin elasticity and reducing the appearance of fine lines and wrinkles by replenishing moisture from within, thereby enhancing the overall rejuvenating effects of the formulation. Typically, HMW Hyaluronic Acid and LMW Hyaluronic Acid each range approximately 0.30% to 0.50% in accordance with embodiments, balancing their roles in surface-level moisture retention and deeper skin hydration, respectively. BioEcolia, included at approximately 0.10% to 0.50%, is known for balancing the skin's microbiota, essential for maintaining skin health. Epsom Salts, included in an embodiment of the formulation at approximately 0.50%, are valued for their detoxifying properties and ability to soothe muscle stiffness, indirectly benefiting skin health by promoting relaxation.

The formulation in embodiments is designed to offer multiple skin health benefits. It aims to improve skin hydration, elasticity, and overall texture. Regular use of the formulation can help to strengthen the skin's barrier function, protect against environmental stressors, and diminish the signs of aging. The formulation is enriched with bioactive compounds that support skin health at a cellular level, promoting a healthier, more youthful appearance.

The preferred embodiment of the moisturizing formulation includes a precise formulation that ensures optimal performance and stability. In various embodiments, the ingredients of the formulation listed herein are approximated and deviate slightly in concentration while retaining the benefits of the moisturizing formulation. The formulation of the preferred embodiment comprises DI Water, constituting 68.10% of the total composition, providing a pure and stable solvent base that enhances the effectiveness of other ingredients. The formulation of the preferred embodiment further comprises Aloe Barbadensis Leaf at a concentration of 0.10%, known for its soothing and hydrating properties. The formulation of the preferred embodiment further comprises glycerin, a potent humectant, is included at 0.90% to draw moisture into the skin, enhancing hydration and barrier function. The formulation of the preferred embodiment further comprises xanthan gum, at a concentration of 0.30%, which serves as a thickening agent, ensuring the formulation maintains a consistent viscosity for easy application. The formulation of the preferred embodiment further comprises Magnesium Sulfate, at a concentration of 0.50%, which acts as a skin conditioner and stabilizes the emulsion. The formulation of the preferred embodiment further comprises Niacinamide at a concentration of 2.00% to improve skin elasticity and reduce discoloration, and Propanediol, at a concentration of 4.00%, which enhances ingredient absorption and provides additional hydration. The formulation of the preferred embodiment further comprises Hydrolyzed Rice Protein, at a concentration of 0.30%, offers conditioning and soothing effects. The formulation of the preferred embodiment further comprises the antioxidant 3-Glyceryl Ascorbate, a potent form of Vitamin C, at a concentration of 10.00%, helping to brighten the skin and promote collagen synthesis. The formulation of the preferred embodiment further comprises Alpha-Glucan Oligosaccharide, at 0.10%, which strengthens the skin's natural barrier, and further comprises Saccharide Isomerate, at a concentration of 2.00%, which provides deep hydration and lasting moisture retention. The formulation of the preferred embodiment further comprises a preservative, consisting of Nipaguard SCE at a concentration of 1.00%, which ensures the product's integrity by preventing microbial growth. The formulation of the preferred embodiment also comprises high molecular weight (HMW) Hyaluronic Acid and low molecular weight (LMW) Hyaluronic Acid, each at concentrations ranging from 0.30% to 0.50%, which provide multi-layered hydration effects. The formulation of the preferred embodiment further comprises BioEcolia, at a concentration within the range of 0.10% to 0.50%, which balances the skin's microbiota, essential for maintaining skin health. The formulation of the preferred embodiment further comprises Epsom Salts, at a concentration of approximately 0.50%, which are included for their detoxifying properties. The formulation of the preferred embodiment further comprises Astragalus Membranaceus Root Extract, at a concentration of 10.00%. This extract enhances the skin's resilience against environmental stressors and aging, providing significant benefits to the formulation's effectiveness. This formulation of the preferred embodiment, as described with specific concentration amounts for each ingredient at a percentage of total composition of the formulation, ensures that the moisturizing formulation not only meets the hydration needs of the skin but also remains effective and stable over time, making it suitable for all skin types, including sensitive skin.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

We claim:

1. A method for producing a moisturizing formulation, comprising the step of:
adding Astragalus Membranaceus Root Extract and 3-Glyceryl Ascorbate to distilled water in a first container;
pre-mixing Xanthan Gum with Glycerin in a second container before adding to the first container to ensure a homogeneous distribution;
mixing the contents of the first container for at least 20 minutes to allow the ingredients to interact and stabilize;
sequentially adding Epsom Salts, HMW Hyaluronic Acid, Vitamin C, BioEcolia, LMW Hyaluronic Acid, and a preservative Nipaguard SCE to the first container;
mixing thoroughly after the addition of each component to ensure complete integration and prevent premature reactions; and
continuing to mix the final combination for at least 30 minutes to ensure proper dispersion of Hyaluronic Acid.

2. The method of claim 1, the adding step further comprising adding an item selected from the group consisting of:
Aloe Barbadensis Leaf, Glycerin, Xanthan Gum, Magnesium Sulfate, Niacinamide, Propanediol, Hydrolyzed Rice Protein, Alpha-Glucan Oligosaccharide, and Saccharide Isomerate
to the first container.

3. A moisturizing formulation comprising:
Astragalus Membranaceus Root Extract at 5%-15% of the total composition,
3-Glyceryl Ascorbate at 5%-15% of the total composition;
and at least one ingredient selected from the group consisting of:
DI Water at approximately 68.10% of the total composition;
Aloe Barbadensis Leaf at approximately 0.10% of the total composition;
Glycerin at approximately 0.90% of the total composition;
Xanthan Gum at approximately 0.30% of the total composition;
Magnesium Sulfate at approximately 0.50% of the total composition;
Niacinamide at approximately 2.00% of the total composition;
Propanediol at approximately 4.00% of the total composition;
Hydrolyzed Rice Protein at approximately 0.30% of the total composition;
Alpha-Glucan Oligosaccharide at approximately 0.10% of the total composition;
Saccharide Isomerate at approximately 2.00% of the total composition;
Nipaguard SCE at approximately 1.00% of the total composition;
HMW Hyaluronic Acid at approximately 0.40% of the total composition;
LMW Hyaluronic Acid at approximately 0.30% of the total composition;
BioEcolia of 0.10%-0.50% of the total composition; and
Epsom Salts at approximately 0.50% of the total composition.
